(19) United States
(12) Patent Application Publication
(10) Pub. No.: US 2003/0054429 A1
(43) Pub. Date: Mar. 20, 2003

(54) Dry analytical element for immunoassay

(75) Inventors: Kentaro Nakamura, Saitama (JP); Kazuya Kawasaki, Saitama (JP); Osamu Seshimoto, Saitama (JP)

Correspondence Address: REED SMITH, LLP, ATTN: PATENT RECORDS DEPARTMENT, 599 LEXINGTON AVENUE, 29TH FLOOR, NEW YORK, NY, 10022-7650, US

(73) Assignee: FUJI PHOTO FILM CO., LTD.

(21) Appl. No.: 10/234,541

(22) Filed: Sep. 3, 2002

(30) Foreign Application Priority Data
Sep. 7, 2001 (JP) 272011/2001

(51) Int. Cl. C12Q 1/54

(52) U.S. Cl. 435/14; 422/56

(57) ABSTRACT

A dry analytical element for immunoassay that is not affected by maltose internally caused by a test sample and has high accuracy is provided. In the analytical element, either (1) fragmentation speed of the maltose to glucose is made larger (a first aspect) or (2) the maltose is removed before it reaches a reagent layer (a second aspect).

### BACKGROUND OF THE INVENTION

1. Field of the Invention

The present invention relates to a dry analytical element for immunoassay to which an enzyme immunoassay method is applied. More particularly, the present invention relates to an immunoassay element for measuring an amount of antigen or antibody, which is an analyte, by detecting glucose generated by a further decomposition process of a product produced by a labeled enzyme, wherein the immunoassay element is arranged so that it is not affected by maltose contained in a test sample.

2. Description of the Related Art

Conventionally, there is an enzyme immunoassay method as one of measuring methods of an analyte in blood. The method bases on the fact that an enzymatic activity is affected by some kind of interference when an antibody and an antigen conjugate each other, and it utilizes an inhibition behavior due to an antigen-antibody conjugation. In general an antigen is labeled with an enzyme, and suppression of the enzymatic activity that is brought about by steric hindrance of bonding of the enzyme to a substrate or alteration of steric structure of the enzyme caused by bonding of an antibody, which is a large molecule, to the antigen.

In clinical diagnosis, in which many test samples are routinely handled, simple and fast analysis and further, automatic operations are desired. From this point of view, a dry analytical element has been proposed. A dry analytical element described in Japanese Patent Laid-open Publication No. H1-321360, for example, contains following (A) to (C) in the same layer or different layers of a multi-layer analytical element: (A) a polymirized antigen (a conjugate of a ligand and a high molecular weight compound), (B) a water-insoluble high molecular weight substrate and (C) a conjugate of an antibody capable of acting on the ligand and an enzyme capable of acting on the substrate. An antigen supplied onto the analytical element by spotting conjugates with the conjugate of the antibody-ligand while competing with the polymerized antigen. A complex of antigen-antibody-enzyme reacts on the water-insoluble high molecular weight substrate to produce a soluble low molecular weight substance. On the other hand, a complex of high molecular weight antigen-antibody-enzyme generated by conjugating to the polymerized antigen cannot show enzymatic activity on the high molecular weight substrate. Therefore, in proportion to increase of the amount of an antigen in a test sample, a product resulted from enzymatic reaction increases. The product is forced to move into a detection layer, and the amount of it is measured basing on an absorption optical density given by a colored chemical group. Thus, the amount of the antigen in the test sample is analyzed.

An analytical element for immunoassay described in Japanese Patent No. 2,576,910 is more improved than the above-described analytical element. In the analytical element, a reagent layer is provided, in which an enzyme that fragments a decomposed product by the labeled enzyme into lower molecular weight is contained. By detecting a lower molecular weight product (glucose), rise in sensitivity is achieved.

When a measuring object is a high molecular weight antigen, the analytical element for immunoassay described in the Japanese Patent No. 2,576,913 can be utilized. The analytical element contains both of (A) a water-insoluble high molecular weight substrate and (B) a conjugate of an antibody capable of acting on a high molecular weight antigen and an enzyme capable of acting on a substrate in the same layer or different layers of a multi-layer analytical element. In the analytical element, a reagent layer is provided that contains a fragmenting enzyme for further fragmenting a decomposed product by the labeled enzyme (glucose oligomer), as is the case with the analytical element for immunoassay described in the Japanese Patent No. 2,576,910, to achieve rise in sensitivity by detecting a fragmented product (glucose).

On the other hand, in these years maltose infusion has been widely performed for feeding transvenously carbohydrate for such a period as prognosis of operation and the like when oral ingestion of nutrition is impossible, or for diabetic patients. In this case, when conventional analytical elements for immunoassay are used, there is a problem such that an error occurs in measurement of CRP concentration.

### SUMMARY OF THE INVENTION

An object of the present invention is to provide an analytical element for immunoassay that is not affected by internally caused maltose in a test sample and has a high accuracy.

Inventors of the invention have concentrated their thoughts to solve the above problem and found that, when conventional analytical elements are used, a small reaction speed at the stage in which maltose is decomposed to glucose gives rate photometry a plus error, resulting in an error in a targeted measurement of CRP concentration.

The invention is accomplished according to the above-described information, and the object of the invention has been achieved by a dry analytical element for immunoassay being applied with either of methods (1) or (2): (1) make fragmentation speed from maltose to glucose larger (a first aspect); (2) remove maltose before it reaches the reagent layer (a second aspect).

### BRIEF DESCRIPTION OF THE DRAWINGS

FIG. 1 is a schematic diagram indicating the basic constitution of a dry analytical element for immunoassay according to the present invention.

FIG. 2 indicates change of reflection optical density with respect to time when a liquid containing maltose (MAL(+)) or not containing maltose (MAL(−)) is spotted onto slides according to the invention described in Examples 1 and 2 and a slide described in Comparative Example 1, respectively.

FIG. 3 indicates relationship between difference of the reflection optical density at 3 minutes and five minutes after spotting a liquid containing a known amount of CRP and the amount of CRP when the liquid is spotted onto slides described in Examples 3 and 4 and a slide described in Comparative Example 2, respectively.

FIG. 4 indicates relationship between difference of the reflection optical density at 3 minutes and five minutes after spotting liquids containing a known amount of CRP and various amount of maltose respectively onto the slides described in Examples 3 and 4 and the slide described in Comparative Example 2, respectively.

### DETAILED DESCRIPTION OF THE INVENTION

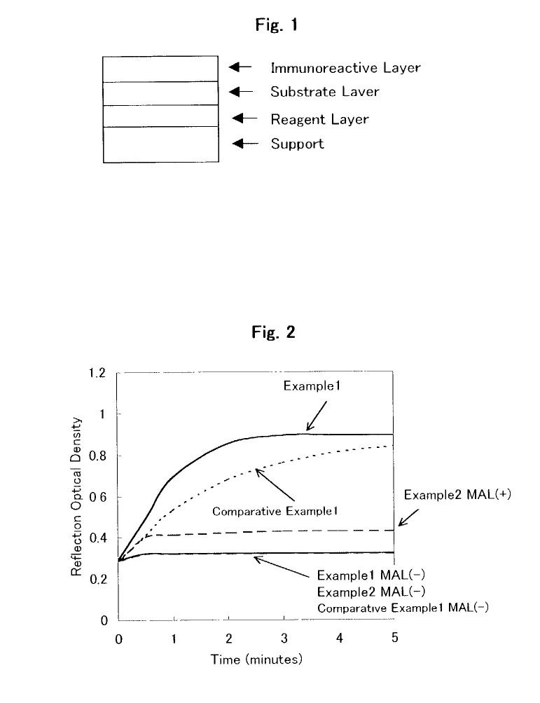

The basic embodiment of the dry analytical element for immunoassay according to the invention is shown in FIG. 1.

The immuno-reactive layer in the embodiment may have facility of spreading the spotted liquid evenly in a horizontal position.

As for the support, any of light impermeable (opaque), light semipermeable (semitransparent) or light permeable (transparent) can be used. In general, a light permeable and water impermeable support is preferable. Preferable materials for the light permeable and water impermeable support are polyethylene terephthalate, polystyrene and the like. In general, an under coating layer is provided on the support or surface of the support is subjected to some treatment so as to become hydrophilic for the purpose of fast adhesion of a hydrophilic layer.

The reagent layer is constituted of a water permeable layer that contains a fragmenting enzyme to fragment glucose oligomer diffused and moved from the substrate layer to glucose, which is a lower molecular weight product. The reagent layer also contains a reagent composition to detect the lower molecular weight product.

The substrate layer is constituted of a water permeable layer, which contains a non-diffusible substrate that is the substrate for an enzyme conjugated to an antibody as a label. The substrate produces a diffusible substance by enzymatic activity remaining in inversely proportional to an amount of a high molecular weight antigen that is the measuring object. As examples of the non-diffusible substrate, carboxymethylated starch, starch, amylose, amlopectin and the like can be listed.

The immuno-reactive layer is a layer containing a kind of molecule necessary for immunoreaction. For example, it contains:

(1) an enzyme-labeled antibody when an amount of an antigen to be detected is analyzed by reacting the antigen to be detected with the enzyme-labeled antibody;

(2) an antibody and an enzyme-labeled antigen when an amount of an antigen to be detected is analyzed by reacting the antigen to be detected and an enzyme-labeled antigen with the antibody;

(3) a polymerized antigen and an enzyme-labeled antibody when an amount of an antigen to be detected is analyzed by reacting the antigen to be detected and the polymerized antigen with the enzyme-labeled antibody;

(4) an enzyme-labeled antigen when an amount of an antibody to be detected is analyzed by reacting the antibody to be detected with the enzyme-labeled antigen; and

(5) an antigen and an enzyme-labeled antibody when an amount of an antibody to be detected is analyzed by reacting the antibody to be detected and the enzyme-labeled antibody with the antigen.

The measuring object of the dry analytical element according to the invention is a high molecular weight antigen that has high molecular weight and an antigen determinant. That is to say, an interferential (depressant) action on enzymatic activity, which is caused by conjugation of a high molecular weight antigen to an enzyme-labeled antibody, is utilized. Therefore, it is preferable that the molecular weight of the high molecular weight antigen is high enough so that it can affect on the enzymatic activity. For example, the analytical element according to the invention displays its greatest force for quantitative analysis of an antigen having molecular weight of not less than 20,000 daltons, and preferably not less than 50,000 daltons.

Any high molecular weight antigen can be analyzed with the analytical element according to the invention if it has such high molecular weight and nature of antigen, that is, an antibody for it can be arranged. For example, there are antigens such that hormones originated from various endocrines, plasma proteins such as immunoglobulin, albumin, ferritin, C-reactive protein (hereinafter, abbreviated as to CRP) and the like, antigens contained in various organs, blood or urine such as virus such as HB antigen and the like, bacteria, α-fetoprotein, carcinoembryonic antigen (CEA) and the like.

Kind of samples containing the high molecular weight antigen is not limited and, for example, blood (whole blood, plasma and serum), lymph fluid, urine and the like are included. It is preferable to previously remove suspended matter, if any, such as blood cell and the like. However, when a suitable filtering layer is provided as the uppermost layer of the analytical element, test samples may be spotted or supplied onto the element as they are.

A non-diffusible substrate contained in the substrate layer is non-diffusible to an aqueous test sample liquid and dose not diffuse as it is into the reagent layer. An enzyme bound to an antibody as a label splits the high molecular weight non-diffusible substrate into a diffusible product, which produces a further low molecular weight product by a fragmenting enzyme. As for such enzymes, splitting enzymes such as producing diffusible oligomers from a non-diffusible substrate consisting of a polymerized body may be included and for example, a carbohydrate hydrolase is listed. Examples of such carbohydrate hydrolase are α-amylase, β-amylase, dextranse and the like. Examples of other hydrolase are cellulase, collagenase, mannase, lipase, ribonuclease and the like.

The fragmenting enzyme contained in the reagent layer fragments the diffusible product produced from the non-diffusible substrate by the enzyme bound to the antibody as a label into a further low molecular weight product, which is capable of being detected. Examples of the fragmenting enzyme include carbohydrate hydrolases, and more specifically α-amylase, β-amylase, dextranase, glucoamylase, α-glucosidase and the like can be listed. A low molecular weight product (glucose) produced by the fragmenting enzyme in the reagent layer can be optically detected by use of known series of detection reagents.

As for the detection method for finally produced glucose, any of following known methods, for example, may be applied: (a) detection method for hydrogen peroxide produced by oxidation of glucose in the presence of glucose oxidase (for example, a method using Trinder reagent described in Ann. Clin. Biochem., 6, 24 (1964) and J. Clin. Pathol., 22, 246 (1969); improved Trinder reagent described in Japanese Patent Laid-open Publication No. S49-50991 (corresponding to U.S. Pat. No 3,886,045), U.S. Pat. No. 3,992,158, Japanese Patent Laid-open Publication No. S55-165356 (corresponding to U.S. Pat. No. 4,292,272) etc.; a reagent containing triaryl-substituted imidazole leuco dye described in Japanese Patent Laid-open Publication Nos. S53-26188 (corresponding to U.S. Pat. No. 4,089,747), S58-45557 etc.; and a reagent containing diaryl-monoaralkyl-substituted imidazole leuco dye described in Japanese Patent Laid-open Publication Nos. S59- 193352 (corresponding to EP 0122641 A), S60-224677 (corresponding to U.S. Pat No. 4,665,023) etc., (b) detection method for NADH generated in the presence of glucose dehydrogenase and NAD; and (c) detection method for glucose-6-phosphate generated in the presence of hexokinase. Among these detection methods, the method in which hydrogen peroxide, which is generated by oxidizing glucose in the presence of glucose oxidase, is detected with peroxidase and leuco dye is most preferable in terms of sensitivity.

In a first embodiment of the invention, the amount of a fragmenting enzyme contained in the above-described reagent layer is adjusted. When the amount of the fragmenting enzyme is small, fragmentation of internal maltose contained in a test sample to glucose takes time, resulting in giving a plus error to rate photometry of main reaction, in which glucose oligomer that is produced by degradation of the non-diffusible substrate is fragmented to glucose by the fragmenting enzyme. Therefore, the fragmenting enzyme should be contained in the reagent layer as much amount as it can fragment the internally caused maltose to glucose in early stages. Thus, to make the amount of the expensive fragmenting enzyme minimum, it is determined experimentally taking the constitution of layers, reaction system, and detection system etc. of the analytical element into consideration. As for usable enzymes, glucoamylase, α-glucosidase and the like are listed. Further, the reaction system above described may be included in a diluting solution for a test sample.

In a second embodiment of the invention, an internally caused maltose is removed with an enzyme before it reaches the reagent layer. For this purpose, it is preferable to provide a layer containing an enzyme that can convert maltose to a substance except for glucose (maltose-removing layer) on upside of the reagent layer. The substrate layer may be made to have this function, or an independent layer may be provided on the substrate layer. Further, the above-mentioned reaction system may be built in a diluting solution for a test sample.

Following substances are contained in the maltose-removing layer:

(1) one selected from the group consisting of glucose amylase, α-glucosidase and maltose phosphorylase, hexokinase and adenosine triphosphate (ATP); or

(2) one selected from the group consisting of glucose amylase, α-glucosidase and maltose phosphorylase, glucose dehydrogenase and NAD.

In the above (1) and (2), when maltose phosphorylase is used, prescription amount of hexokinase and ATP or that of glucose dehydrogenase and NAD may be reduced.

The dry analytical element according to the present invention may have the same layer structure as that of various known dry analytical elements. The element may be constituted of a multilayer including, along with the substrate layer, reagent layer and immuno-reactive layer, a support, a spreading layer, a detection layer, a light-shielding layer, an adhesive layer, a water absorption layer, an undercoating layer and other layers. Examples of such an analytical element include those disclosed in Japanese Patent Laid-open Publication Nos. S49-53888 (corresponding to U.S. Pat. No. 3,992,158), S51-40191 (corresponding to U.S. Pat. No. 4,042,335), S55-164356 (corresponding to U.S. Pat. No. 4,292,272), S61-4959 (corresponding to EP 0166365 A) and H10-300751.

The reagent layer may be constituted of different multiple layers. A water absorption lay may be provided between the support and the reagent layer or detection layer. A filtration layer may be provided between respective layers. Further, a spreading layer may be provided on the immuno-reactive layer; or the immuno-reactive layer may be given spreading behavior to function as the spreading layer.

Hereinafter, the invention will be described in more detail according to examples, however the invention will not be limited to the examples.

### EXAMPLES

### Example 1

### A First Embodiment

On a colorless and transparent polyethylene terephthalate sheet of 180 μm in thickness provided with a gelatin undercoating layer (support), a reagent solution containing a crosslinking agent was coated, followed by drying, to form a reagent layer so that respective components had the coverage as set forth bellow.

| | | |
|---|---|---|
| Alkali-processed gelatin | 14.0 | g/m² |
| Nonylphenoxypolyethoxyethanol | 1.0 | g/m² |
| Glucose oxidase | 6000 | U/m² |
| Peroxidase | 12000 | U/m² |
| Glucoamylase | 25000 | U/m² |
| 2-(4-hydroxy-3,5-dimethoxyphenyl-4- | 0.5 | g/m² |
| [4-(dimethylamino)phenyl]-5-phenethylimidazole | | |
| (leuco dye) acetate | | |
| Bis[(vinylsulfonylmethylcarbonyl)amino] methane | 0.2 | g/m² |

Next, on the above-described reagent layer, an aqueous solution containing following reagents was coated so that respective components had the coverage as set forth bellow.

| | |
|---|---|
| Alkali-processed gelatin | 10.2 g/m² |
| Nonylphenoxypolyethoxyethanol | 0.5 g/m² |
| Bis[(vinylsulfonylmethylcarbonyl)amino] methane | 0.1 g/m² |

Next, on the above-described sheet, an aqueous solution containing following reagents was coated and dried so that respective components had the coverage as set forth bellow to

| | |
|---|---|
| Hydroxypropylcellulose | 4.7 g/m² |
| Carboxymethylated starch | 3.5 g/m² |
| PIPES | 0.9 g/m² |
| Mannitol | 2.3 g/m² |
| Nonylphenoxypolyethoxyethanol | 1.2 g/m² |

Next, water was supplied on the entire surface of the film in a feed rate of about 60 g/m² to wet, then a tricot knitted fabric formed by knitting 50 denier polyethylene terephthalate spun yarn with 36 gauge was laminated with light pressure and dried. On the fabric, ethanol was coated so as to be 200 g/m² (=OC1 coating) and dried, then an ethanol solution was coated so that the coated layer would contain respective components in following amount and be 5 μm in thickness after drying (=OC2 coating) and dried to prepare an integrated multilayer analytical element.

| | |
|---|---|
| Polyvinylpyrrolidone | 5.6 g/m² |
| Nonylphenoxypolyethoxyethanol | 0.2 g/m² |

An integrated multilayer analytical element thus formed was cut into a square chip of 12 mm×13 mm and was housed in a slide casing (described in Japanese Patent Laid-open Publication S57-63452). By this means a dry slide 1 for CRP analysis according to a first embodiment of the invention was prepared.

### Example 2

### A Second Embodiment

On a colorless and transparent polyethylene terephthalate sheet of 180 μm in thickness provided with a gelatin undercoating layer, a reagent solution containing a crosslinking agent was coated, followed by drying, to form a reagent layer so that respective components had the coverage as set forth bellow.

| | | |
|---|---|---|
| Alkali-processed gelatin | 14.0 | g/m² |
| Nonylphenoxypolyethoxyethanol | 1.0 | g/m² |
| Glucose oxidase | 6000 | U/m² |
| Peroxidase | 12000 | U/m² |
| Glucoamylase | 5000 | U/m² |
| 2-(4-hydroxy-3,5-dimethoxyphenyl-4- | 0.5 | g/m² |
| [4-(dimethylamino)phenyl]-5-phenethylimidazole | | |
| (leuco dye) acetate | | |
| Bis[(vinylsulfonylmethylcarbonyl)amino] methane | 0.2 | g/m² |

Next, on the above-described reagent layer, an aqueous solution containing following reagents were coated so that respective components had the coverage as set forth bellow.

| | |
|---|---|
| Alkali-processed gelatin | 10.2 g/m² |
| Nonylphenoxypolyethoxyethanol | 0.5 g/m² |
| Bis[(vinylsulfonylmethylcarbonyl)amino] methane | 0.1 g/m² |

Next, on the above-described sheet, an aqueous solution containing following reagents was coated and dried so that respective components had the coverage as set forth bellow to form a substrate layer.

| | | |
|---|---|---|
| Hydroxypropylcellulose | 4.7 | g/m² |
| Carboxymethylated starch | 3.5 | g/m² |
| PIPES | 0.9 | g/m² |
| Mannitol | 2.3 | g/m² |
| Nonylphenoxypolyethoxyethanol | 1.2 | g/m² |
| Glucoamylase | 25000.0 | U/m² |
| Glucokinase (hexokinase Type IV) | 5000.0 | U/m² |
| ATP (adenosine triphosphate) | 2.0 | g/m² |

Next, water was supplied on the entire surface of the sheet in a feed rate of about 60 g/m² to wet, then a tricot knitted fabric formed by knitting 50 denier polyethylene terephthalate spun yarn with 36 gauge was laminated with light pressure and dried.

On the fabric, ethanol was coated so as to be 200 g/m² (=OC1 coating) and dried, then all ethanol solution was coated so that the coated layer would contain respective components in following amount and be 5 μm in thickness after drying (=OC2 coating) and dried to prepare an integrated multilayer analytical element.

| | |
|---|---|
| Polyvinylpyrrolidone | 5.6 g/m² |
| Nonylphenoxypolyethoxyethanol | 0.2 g/m² |

The integrated multilayer analytical element thus formed was cut into a square chip of 12 mm×13 mm and was housed in a slide casing (described in Japanese Patent Laid-open Publication S57-63452). By this means a dry slide 2 for CRP analysis according to a second embodiment of the invention was prepared.

### Comparative Example 1

A slide 3 was prepared as a Comparative Example in the same manner as the slide 1 prepared in Example 1 except for replacing 25000 U/M² of glucoamylase in the reagent layer with 5000 U/m².

### Performance Evaluation Test 1

To slides 1, 2 and 3, 10 μL of 50 mM MES (2-(N-morpholino) eathanesulfonic acid monohydrate) buffer solution (pH 6) was dotted, respectively. On the other hand, 10 μL of a solution prepared by 21-time diluting an aqueous solution of 400 mg/dl maltose with 50 mM MES (2-(N-morpholino) eathaniesulfonic acid monohydrate) buffer solution (pH 6) was dotted to slides 1, 2 and 3, respectively. After the dotting, each slide was kept at 37° C. and time course measurement of reflection optical density of each slide was performed at 650 nm through the support. Alteration of the reflection optical density in terms of elapse after the dotting is shown in FIG. 2.

As for the slide 3 of Comparative Example 1, a significant increase was observed in the reflection optical density after dotting the maltose-containing solution (in FIG. 2, Comparative Example 1 (MAL (+)). It was on the rise even after 2-minute elapse after the dotting and the reflection optical density increased. On the other hand, as for the slide 1 of Example 1, though a significant increase was observed in the reflection optical density just after the dotting (in FIG. 2, Example 1 (MAL (+)), the reflection optical density was an approximate constant value after elapse of two minutes from the dotting. Here, MAL (−) means data when the solution that is not containing maltose was dotted, which shows the optical density of background.

These results are interpreted as follows: as for the slide 3, since fragmentation reaction of maltose, which is contained in the dotted test sample and moves from the substrate layer to the reagent layer, to glucose by glucoamylase in the reagent layer progresses slowly, the rise of the reflection optical density continues; on the other hand as for the slide 1, since fragmentation speed of maltose to glucose rises by increase of the addition amount of glucoamylase, the fragmentation terminates within two minutes after the dotting of the test sample, resulting in no rise of the reflection optical density after that period of time.

As for the slide 2 of the Example 2, though the reflection optical density rose for about 20 seconds after dotting the maltose containing solution, it reached plateau and kept a constant value (in FIG. 2, Example 2 (MAL (+)) after that period of time. This result is interpreted as follows: maltose in the solution moves, just after the dotting, to the reagent layer along with infiltration of the solution before being removed in the maltose-removing layer, resulting in increase in the reflection optical density for some period of time after the dotting; however, when the infiltration up to the reagent layer terminates, thereafter maltose is fragmented to glucose, which is further consumed by glucokinase and the maltose in the solution scarcely moves to the reagent layer.

### Example 3

### The First Embodiment

A multilayer analytical element was prepared in the same manner as Example 1. On the tricot knitted fabric of it, which was a spreading layer, ethanol was coated so as to be 200 g/m² (=OC1 coating), then an ethanol solution containing following reagents was coated so as to be a following covering amount and 5 μm in dry thickness (=OC2 coating) and was dried to prepare an integrated multilayer analytical element.

| | | |
|---|---|---|
| Amylase-labeled anti-C-reactive protein mouse antibody | 14.0 | KU/m² |
| Anti-C-reactive protein mouse second antibody | 6.2 | mg/m² |
| Polyvinylpyrrolidone | 5.6 | g/m² |
| Nonylphenoxypolyethoxyethanol | 0.2 | g/m² |

The integrated multilayer analytical element thus formed was cut into a square chip of 12 mm×13 mm and was housed in a slide casing (described in Japanese Patent Laid-open Publication S57-63452). By this means a dry slide 4 for CRP analysis according to the first embodiment of the invention was prepared.

### Example 4

### The Second Embodiment

A multilayer analytical element was prepared in the same manner as Example 2. On the tricot knitted fabric of it, which was a spreading layer, ethanol was coated so as to be 200 g/m² (=OC1 coating), then an ethanol solution containing following reagents was coated so as to be a following covering amount and 5 μm in dry thickness (=OC2 coating) and was dried to prepare an integrated multilayer analytical element.

| | | |
|---|---|---|
| Amylase-labeled anti-C-reactive protein mouse antibody | 14.0 | KU/m² |
| Anti-C-reactive protein mouse second antibody | 6.2 | mg/m² |
| Polyvinylpyrrolidone | 5.6 | g/m² |
| Nonylphenoxypolyethoxyethanol | 0.2 | g/m² |

The integrated multilayer analytical element thus formed was cut into a square chip of 12 mm×13 mm and was housed in a slide casing (described in Japanese Patent Laid-open Publication S57-63452). By this means a dry slide 5 for CRP analysis according to the second embodiment of the invention was prepared.

### Comparative Example 2

A slide 6 was prepared as a comparative example in the same manner as the slide 4 prepared in Example 3 except for replacing 25000 U/m² of glucoamylase in the reagent layer with 5000 U/M².

### Performance Evaluation Test 2

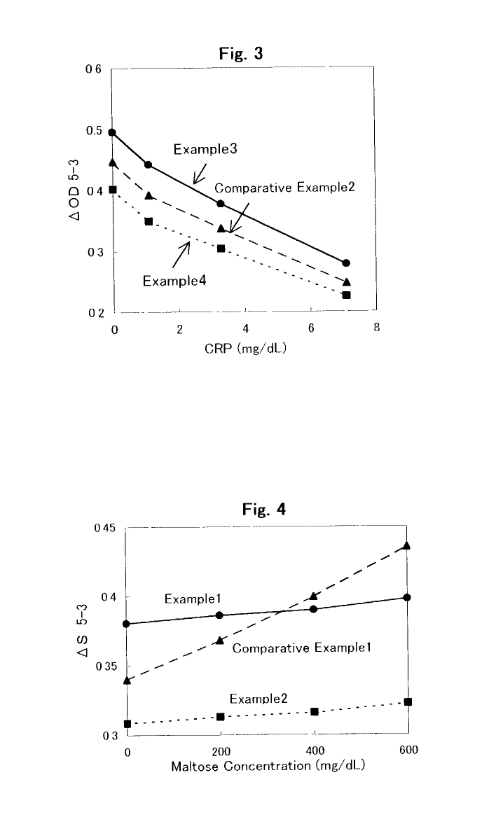

To the slide 4 of Example 3, the slide 5 of Example 4 and the slide 6 of Comparative Example 2, 10 μL of 50 mM MES buffer solution (pH 6) containing a known amount of CRP was dotted, respectively. After the dotting, each slide was kept at 37° C. and reflection optical density of each slide was measured with visible light having the central wave length of 650 nm through the PET support. Difference between the reflection optical density at 3 minutes and 5 minutes after the dotting (Δ OD5-3) for each slide is shown in FIG. 3.

From calibration curves in FIG. 3, it is obvious that the slide 5 that is provided with the maltose-removing layer is capable of performing quantitative determination of CRP with high accuracy, as are the case with the slide 6 of the comparative example 2 and the slide 4 of example 3 that are not provided with it. This shows that the maltose-removing layer gives no adverse effect on detection sensitivity of quantitative determination of CPR. Further, since reaction speed on disaccharide or trisaccharide generated by decomposition of carboxymethylated starch, which is the substrate, increases as to the slide 4 of the Example 3 comparing to the slide 6 of Comparative Example 2, the slide 4 shows more extended calibration curve and will be expected to have a further higher measurement accuracy comparing to the slide 6.

### Performance Evaluation Test 3

To the slides 4, 5 and 6, 10 μL of 50 mM MES buffer solution (pH 6) containing a known certain amount of CRP and various concentrations of maltose was dotted, respectively. After the dotting, each slide was kept at 37° C. and reflection optical density of each slide was measured at 650 nm through the PET support. Further, the above-described reflection optical density was converted to an amount of dye using a previously provided conversion equation that related the reflection optical density to the amount of produced dye. Thus difference of the amounts of dye produced at 3 minutes and 5 minutes after the dotting (ΔS 5-3), respectively, was calculated. As shown in FIG. 4, as for the conventional slide 6 of Comparative Example 2, ΔS 5-3 increased with increase of concentration of maltose in the test sample liquid. On the contrary, as for the slide 4 of Example 3, in which the amount of glucoamylase in the reagent layer was increased, the value ΔS 5-3 was scarcely affected even when maltose was contained in the test sample liquid. Also, as for the slide 5 of Example 4 that is provided with the maltose-removing layer was scarcely affected.

These results clearly specify that the dry analytical element according to the invention can perform an immunoassay with high accuracy even when maltose concentration varies depending on test samples.

### Effect of the Invention

The present invention can provide an analytical element for immunoassay that is not affected by internally caused maltose in a test sample and has high accuracy.

What is claimed is:

1. A dry analytical element for immunoassay provided with at least a reagent layer containing an enzyme that decomposes maltose to glucose, a water permeable substrate layer containing a non-diffusible substrate, an immunoreactive layer containing a kind of molecule necessary for immunoreaction on a support in this order, wherein a content of the enzyme that decomposes maltose to glucose is an amount capable of decomposing maltose internally caused by a test sample to glucose within less than three minutes.

2. A dry analytical element for immunoassay provided with at least a reagent layer containing an enzyme that decomposes maltose to glucose, a water permeable substrate layer containing a non-diffusible substrate, an immunoreactive layer containing a kind of molecule necessary for immunoreaction on a support in this order, wherein a layer that removes maltose internally caused by a test sample is further provided on upside of the reagent layer.
